(19) United States
(12) Patent Application Publication
(10) Pub. No.: US 2022/0199221 A1
(43) Pub. Date: Jun. 23, 2022

(54) Method and Apparatus to Deliver Therapeutic Energy to a Patient Using Multi-Objective Optimization as a Function of a Patient's Quality of Care

(71) Applicant: Varian Medical Systems, Inc., Palo Alto, CA (US)

(72) Inventors: Deepak Khuntia, Los Altos, CA (US); Corey E. Zankowski, Cupertino, CA (US); Paritosh Ambekar, Redwood City, CA (US); Alexander E. Maslowski, Peachtree City, GA (US)

(21) Appl. No.: 17/129,050

(22) Filed: Dec. 21, 2020

(51) Int. Cl. G16H 20/40; A61N 5/02; A61N 5/10; A61F 7/00

(52) U.S. Cl. CPC G16H 20/40; A61N 5/02; A61F 2007/0093; A61F 7/00; A61N 2005/1074; A61N 5/1031

(57) ABSTRACT

These teachings include accessing energy dosing information along with at least one quality-of-care model that correlates at least one categorical energy-based treatment patient quality-of-care outcome with at least one resultant energy-based treatment description. The model can be created via probabilistic mapping that maps patient impact information to dose impartation information to infer non-biological impact to a patient. A patient treatment plan can be optimized for a particular patient as a function of the foregoing information to provide corresponding resultant benefit trade-of evaluation information. This benefit trade-off evaluation information can be displayed to a user to permit the user to explore the benefit trade-off evaluation information to thereby identify a resultant energy-based treatment plan having a selected balance between dosing a treatment target with energy and a quality-of-care impact on the particular patient.

### TECHNICAL FIELD

These teachings relate generally to treating a patient's planning target volume with energy pursuant to an energy-based treatment plan and more particularly to optimizing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called energy-based treatment plan often serves in the foregoing regards.

An energy-based treatment plan such as a radiation treatment plan typically comprises specified values for each of a variety of treatment-platform parameters during each of a plurality of sequential fields. Treatment plans for radiation treatment sessions are often generated through a so-called optimization process. As used herein, “optimization” will be understood to refer to improving a candidate treatment plan without necessarily ensuring that the optimized result is, in fact, the singular best solution. Such optimization often includes automatically adjusting one or more treatment parameters (often while observing one or more corresponding limits in these regards) and mathematically calculating a likely corresponding treatment result to identify a given set of treatment parameters that represent a good compromise between the desired therapeutic result and avoidance of undesired collateral effects.

Unfortunately, existing optimization techniques do not necessarily address all potential needs for all potential patients in all potential application settings. As one example in these regards, typical existing optimization approaches do not readily capture, reflect, or account for any of a variety of quality-of-care concerns such as financial impact to the patient, toxicity impact to the patient, mortality impact to the patient, short-term physiological side effects experienced by the patient, or quality-adjusted life-years impact to the patient.

### BRIEF DESCRIPTION OF THE DRAWINGS

The above needs are at least partially met through provision of the method and apparatus to facilitate generating a deliverable therapeutic energy-based treatment plan described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings; and

FIG. 3 comprises an illustrative screenshot as configured in accordance with various embodiments of these teachings.

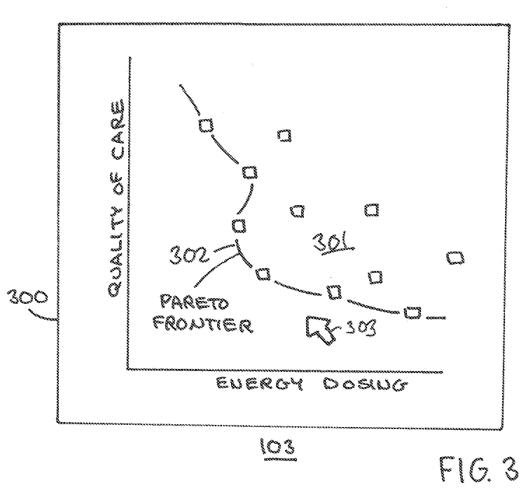

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, these various embodiments serve to facilitate optimizing a patient treatment plan to administer therapeutic energy to a particular patient. These teachings will accommodate a variety of therapeutic energies including, but not limited to, ionizing radiation, microwave energy, and thermal energy.

By one approach these teachings include accessing energy dosing information along with at least one quality-of-care model that correlates at least one categorical energy-based treatment patient quality-of-care outcome with at least one resultant energy-based treatment description. The aforementioned resultant energy-based treatment description can comprise, for example, a description of at least one of energy dose distribution in the treatment target and at least one computed tomography image.

The energy dosing information can comprise, by one approach and at least in part, an energy dosing objective for a treatment target and an energy dosing objective for at least one organ-at-risk. These teachings will accommodate a variety of categorical energy-based treatment patient quality-of-care outcomes including, but not limited to, financial impact to the particular patient, toxicity impact to the particular patient, mortality impact to the particular patient, short-term physiological side effects experienced by the patient, and quality-adjusted life-years impact to the particular patient.

The aforementioned at least one quality-of-care model can comprise, for example, a model created via probabilistic mapping that maps patient impact information to dose impartation information to infer non-biological impact to a patient. Those skilled in the art will appreciate that artificial intelligence techniques can be applied to accomplish that probabilistic mapping.

These teachings then provide for optimizing a patient treatment plan for a particular patient as a function of the energy dosing information and the at least one quality-of-care model using multi-objective optimization to provide corresponding resultant benefit trade-of evaluation information.

By one approach these teachings then provide for displaying to a user at least some of the benefit trade-off evaluation information via an interactive user interface. So configured, the user can explore the benefit trade-off evaluation information to thereby identify a resultant energy-based treatment plan having a selected balance between dosing a treatment target with energy and a quality-of-care impact on the particular patient. By one approach the foregoing can include displaying a corresponding Pareto frontier having user-selectable elements that each represent a potentially optimum solution.

As one example these teachings can provide for radiating a treatment target in a patient during a radiation treatment session with a particular radiation treatment platform having a moving source of radiation and using a radiation treatment plan developed per the foregoing teachings. These teachings will then accommodate operating the aforementioned particular radiation treatment platform as a function of the optimized radiation treatment plan to administer therapeutic radiation to the particular patient.

So configured, these teachings present a way to optimize an energy-based treatment plan as a function, at least in part, of metrics that directly describe any of a variety of quality-of-care patient parameters. In particular, these teachings can provide a user with a mechanism for exploring benefit trade-off evaluation information to thereby better facilitate balancing desired physiological outcomes (such as tumor ablation) against one or more optimal biological/financial impacts to the patient.

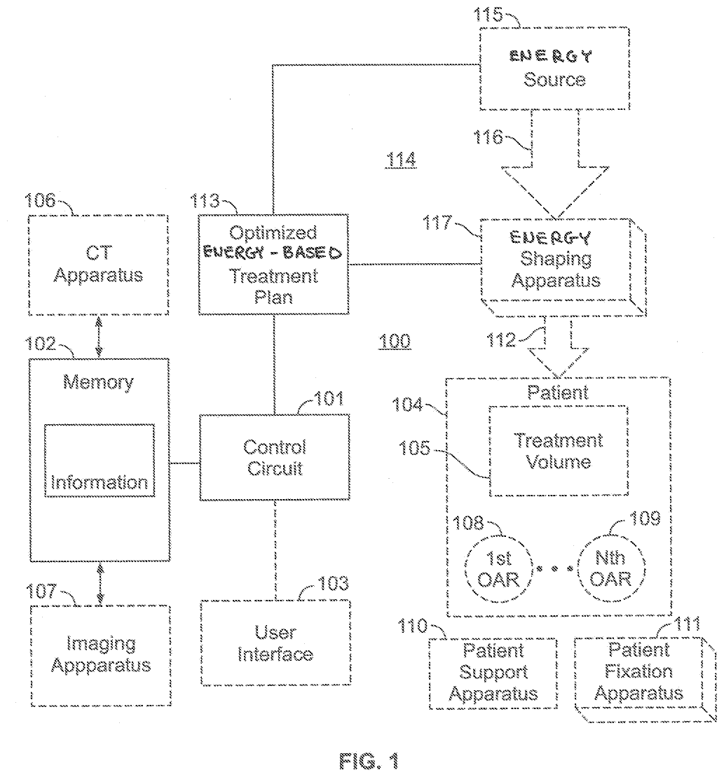

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101).

In addition to information such as energy dosing information and one or more quality-of-care models as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan 113 (such as, for example, an optimized radiation treatment plan). This energy-based treatment plan 113 typically comprises specified values for each of a variety of treatment-platform parameters during each of a plurality of sequential exposure fields. In this case the energy-based treatment plan 113 is generated through an optimization process. Various automated optimization processes specifically configured to generate such an energy-based treatment plan are known in the art. As the present teachings are not overly sensitive to any particular selections in these regards, further elaboration in these regards is not provided here except where particularly relevant to the details of this description.

By one approach the control circuit 101 can operably couple to an energy-based treatment platform 114 that is configured to deliver therapeutic energy 112 to a corresponding patient 104 in accordance with the optimized energy-based treatment plan 113. These teachings are generally applicable for use with any of a wide variety of energy-based treatment platforms/apparatuses.

In a typical application setting the energy-based treatment platform 114 will include an energy source 115 such as a source of ionizing radiation, a source of microwave energy, a source of heat energy, and so forth.

By one approach this energy source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the energy source 115 along that arcuate pathway, and may accordingly control when the energy source 115 starts moving, stops moving, accelerates, de-accelerates, and/or a velocity at which the energy source 115 travels along the arcuate pathway.

As one illustrative example, the energy source 115 can comprise, for example, a radio-frequency (RF) linear particle accelerator-based (linac-based) x-ray source, such as the Varian TrueBeam or Halcyon linear accelerator. The linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the energy source 115, and one or more energy-shaping apparatuses 117 (for example, beam-shaping apparatuses such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

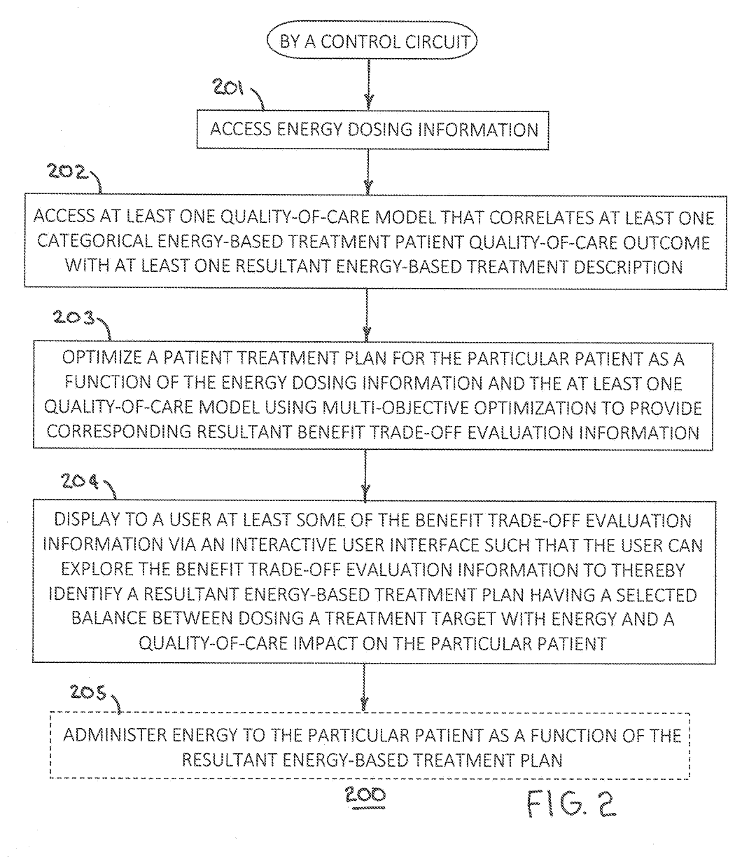

Referring now to FIG. 2, a process 200 that can be carried out, for example, in conjunction with the above-described application setting (and more particularly via the aforementioned control circuit 101) will be described. Generally speaking, this process 200 serves to facilitate dosing a treatment target (105) in a patient (104) during an energy-based treatment session with an energy-based treatment platform (114) having a moving source of energy (115) using an optimized energy-based treatment plan (113).

At block 201, this process 200 can provide for the control circuit 101 accessing energy dosing information from, for example, the aforementioned memory 102. The specific energy dosing information can depend upon the type of energy to be therapeutically applied. Specific temperatures can be identified when applying thermal energy (such as cryotherapeutic energy) and specific frequencies and magnitudes can be identified when applying microwave energy. When applying ionizing radiation, this information can comprise, for example, a minimum radiation dosing objective for a patient's treatment target (such as a tumor) and a maximum radiation dosing for one or more organs-at-risk in the patient.

At block 202, this process can provide for the control circuit 101 accessing at least one quality-of-care model from, for example, the aforementioned memory 102. Each such quality-of-care model can be configured to correlate at least one categorical energy-based treatment patient quality-of-care outcome with at least one resultant energy-based treatment description. (The aforementioned resultant energy-based treatment description can vary with the needs of the application setting. By one approach, this description can comprise a description of at least one of, for example, energy dose distribution in the treatment target (and/or in one or more organs-at-risk) and/or at least one computed tomography image.)

These teachings are highly flexible in practice and will accommodate any of a variety of categorical energy-based treatment patient quality-of-care outcomes. As one example in these regards, the categorical energy-based treatment patient quality-of-care outcome can represent financial impact to the particular patient. By one approach, this financial impact can account for the direct and incidental costs associated with the patient treatment plan itself. By another approach, in lieu the foregoing or in combination therewith, this financial impact can account for follow-on costs that are typically experienced by patients who undergo such treatments (including such things as long term care, specialized housing or dietary requirements, counseling and/or mental or physical therapy, and so forth).

As another example in these regards, the categorical energy-based treatment patient quality-of-care outcome can represent toxicity impact to the particular patient. This toxicity impact can represent negative quality-of-life issues experienced by patients who undergo the patient treatment plan as a result of collateral toxicity associated with the treatment. Examples in these regards include, but are not limited to, dietary difficulties and/or changes, mobility challenges, cognitive challenges, chronic pain, and so forth.

As yet another example in these regards, the categorical energy-based treatment patient quality-of-care outcome can represent mortality impact to the particular patient. Examples include, but are not limited to, a diminution of expected lifetime and/or an increased susceptibility to death by particular causes such as organ failure, accident, cognitive mishap, and so forth.

As yet another example in these regards, the categorical energy-based treatment patient quality-of-care outcome can represent short-term physiological side effects likely to be experienced by the patient. “Short-term” can vary with the application setting, with relevant ranges including, for example, six hours, twenty-four hours, two days, five days, one week, one month, three months, and the like. Examples of such side effects can include fever, bleeding, and so forth.

And as yet another example in these regards, the categorical energy-based treatment patient quality-of-care outcome can represent quality-adjusted life-years (QALY) impact to the particular patient. Those skilled in the art will recognize that the latter constitutes a generic measure of disease burden, including both the quality and the quantity of life lived. By one approach, such a parameter assumes that health is a function of length of life and quality of life and combines these values into a single index number. To determine QALYs, one can therefore multiply the utility value associated with a given state of health by the years lived in that same state of health. For example, a year of life lived in perfect health is worth 1 QALY (1 year of life×1 utility value). Accordingly, a year of life lived in a state of less than perfect health is worth less than 1 QALY. For example, 1 year of life lived in a situation with impaired utility metricized as 0.5 leads to the calculation 1 year×0.5 to yield the result 0.5 QALY. Death is assigned a value of 0 QALYs, and in some circumstances it is possible to accrue negative QALYs to reflect health states deemed worse than being dead.

The aforementioned quality-of-care model can comprise, by one approach, a model created via probabilistic mapping that maps patient impact information (for example, as described above) to dose impartation information to thereby infer non-biological impact to a patient. These teachings will accommodate developing a model via probabilistic mapping by use of artificial intelligence. Artificial intelligence models that parameterize patient and dose impartation to infer biological impact presently exist. Such approaches can be leveraged here to instead create a model that parameterizes patient and dose impartation to infer the kinds of patient impact that are described herein. As such techniques are known in the art, further elaboration is not provided here for the sake of brevity.

In any event, at block 203 this process 200 provides for optimizing a patient treatment plan for the particular patient as a function of the aforementioned energy dosing information and the at least one quality-of-care model using multi-objective optimization to provide corresponding resultant benefit trade-off evaluation information. Multi-objective optimization (also known as multi-criteria optimization, multi-objective programming, vector optimization, multi-attribute optimization, or Pareto optimization) is an area of multiple-criteria decision-making involving more than one objective function to be optimized simultaneously with respect to another. Multi-objective optimization can provide useful results in an application setting where there are conflicting trade-offs between two or more objectives.

For a nontrivial multi-objective optimization problem, there does not usually exist a single solution that simultaneously optimizes each objective. In that case, the objective functions can be said to be conflicting, and there exists a (possibly infinite) number of Pareto optimal solutions. Without additional subjective preference information, all Pareto optimal solutions may be considered equally good. The goal may be to find a representative set of Pareto optimal solutions and/or to quantify the trade-offs in satisfying the different objectives, and/or to find a single solution that satisfies the subjective preferences of a human decision maker.

Accordingly, at block 204 of this process, these teachings provide for displaying to a user at least some of the benefit trade-off evaluation information via, for example, the above-described user interface 103. The user can then explore the benefit trade-off evaluation information to thereby identify a resultant energy-based treatment plan having a selected balance between dosing a treatment target with energy and quality-of-care for the particular patient. In this illustrative example the benefit trade-off evaluation information 301 includes a displayed so-called Pareto frontier 302. Those skilled in the art will understand that a Pareto frontier constitutes the set of all Pareto efficient allocations that pertain to the current inquiry. By presenting this Pareto frontier 302, this process 200 can present essentially or literally all of the potentially optimal solutions, and the user can then explore this frontier and make focused tradeoffs within this constrained set of parameters, rather than needing to consider the full ranges of corresponding parameters.

The user can interact with this display 300 using a modality of choice. When the display 300 comprises a touch screen display the user may simply tap points of potential interest. By another approach, in lieu of the foregoing or in combination therewith, the user may manipulate an on-screen cursor 303 to select points of interest.

This process 200 can optionally include, as illustrated at optional block 205, then operating the particular energy-based treatment platform 114 as a function of the optimized energy-based treatment plan 113 to administer energy to the particular patient 104.

So configured, these teachings can improve the quality of energy-based treatment plans by directly linking how such plans are optimized against real quality-of-care impact to the patient.

Those skilled in the art will recognize that a wide variety of modifications, alterations, and combinations can be made with respect to the above described embodiments without departing from the scope of the invention. Accordingly, such modifications, alterations, and combinations are to be viewed as being within the ambit of the inventive concept.

What is claimed is:

1. A method for optimizing a patient treatment plan to administer therapeutic energy to a particular patient, the method comprising:
by a control circuit:
accessing energy dosing information;
accessing at least one quality-of-care model that correlates at least one categorical energy-based treatment patient quality-of-care outcome with at least one resultant energy-based treatment description;
optimizing a patient treatment plan for the particular patient as a function of the energy dosing information and the at least one quality-of-care model using multi-objective optimization to provide corresponding resultant benefit trade-off evaluation information;
displaying to a user at least some of the benefit trade-off evaluation information via an interactive user interface such that the user can explore the benefit trade-off evaluation information to thereby identify a resultant energy-based treatment plan having a selected balance between dosing a treatment target with energy and a quality-of-care impact on the particular patient.

2. The method of claim 1 wherein the energy dosing information comprises, at least in part, an energy dosing objective for a treatment target and an energy dosing objective for at least one organ-at-risk.

3. The method of claim 1 wherein the therapeutic energy comprises at least one of:
ionizing radiation;
microwave energy;
cryotherapeutic energy.

4. The method of claim 1 wherein the at least one categorical energy-based treatment patient quality-of-care outcome comprises at least one of:
financial impact to the particular patient;
toxicity impact to the particular patient;
mortality impact to the particular patient; and
quality-adjusted life-years impact to the particular patient.

5. The method of claim 1 wherein the at least one resultant energy-based treatment description comprises a description of at least one of:
energy dose distribution in the treatment target; and
at least one computed tomography image.

6. The method of claim 1 wherein the at least one quality-of-care model comprises a model created via probabilistic mapping that maps patient impact information to dose impartation information to infer non-biological impact to a patient.

7. The method of claim 6 wherein the patient impact information comprises, at least in part, financial information.

8. The method of claim 6 wherein the patient impact information comprises, at least in part, mortality information.

9. The method of claim 6 wherein the patient impact information comprises, at least in part, quality-adjusted life-years impact information.

10. The method of claim 1 further comprising:
administering energy to the particular patient as a function of the resultant energy-based treatment plan.

11. An apparatus for optimizing an energy-based treatment plan to administer therapeutic energy to a particular patient, the apparatus comprising:
memory having stored therein:
energy dosing information for the particular patient; and
at least one quality-of-care model that correlates at least one categorical energy-based treatment patient quality-of-care outcome with at least one resultant energy-based treatment description;
an interactive user interface; and
a control circuit operably coupled to the memory and the interactive user interface and being configured to:
access the energy dosing information;
access the at least one quality-of-care model that correlates at least one categorical energy-based treatment patient quality-of-care outcome with at least one resultant energy-based treatment description;
optimize an energy-based treatment plan for the particular patient as a function of the energy dosing information and the at least one quality-of-care model using multi-objective optimization to provide corresponding resultant benefit trade-off evaluation information;
display to a user at least some of the benefit trade-off evaluation information via the interactive user interface such that the user can explore the benefit trade-off evaluation information to thereby identify a resultant energy-based treatment plan having a selected balance between dosing a treatment target with energy and a quality-of-care impact on the particular patient.

12. The apparatus of claim 11 wherein the energy dosing information comprises, at least in part, an energy dosing objective for a treatment target and an energy dosing objective for at least one organ-at-risk.

13. The apparatus of claim 11 wherein the therapeutic energy comprises at least one of:
ionizing radiation;
microwave energy;
cryotherapeutic energy.

14. The apparatus of claim 11 wherein the at least one categorical energy-based treatment patient quality-of-care outcome comprises at least one of:
financial impact to the particular patient;
toxicity impact to the particular patient;
mortality impact to the particular patient;
short-term physiological side effects; and
quality-adjusted life-years impact to the particular patient.

15. The apparatus of claim 11 wherein the at least one resultant energy-based treatment description comprises a description of at least one of:
energy dose distribution in q treatment target; and
at least one computed tomography image.

16. The apparatus of claim 11 wherein the at least one quality-of-care model comprises a model created via probabilistic mapping that maps patient impact information to dose impartation information to infer non-biological impact to a patient.

17. The apparatus of claim 16 wherein the patient impact information comprises, at least in part, financial information.

18. The apparatus of claim 16 wherein the patient impact information comprises, at least in part, mortality information.

19. The apparatus of claim 16 wherein the patient impact information comprises, at least in part, quality-adjusted life-years impact information.

20. The apparatus of claim 11 further comprising:
a treatment platform configured to administer energy to the particular patient as a function of the resultant energy-based treatment plan.
